(19) United States
(12) Patent Application Publication
(10) Pub. No.: US 2003/0054509 A1
(43) Pub. Date: Mar. 20, 2003

(54) Method for producing fats or oils

(75) Inventors: Inmok Lee, Decatur, IL (US); Ronald T. Sleeter, Decatur, IL (US)

Correspondence Address: STERNE, KESSLER, GOLDSTEIN & FOX PLLC, 1100 NEW YORK AVENUE, N.W., SUITE 600, WASHINGTON, DC, 20005-3934, US

(73) Assignee: Archer-Daniels-Midland Company

(21) Appl. No.: 10/100,188

(22) Filed: Mar. 19, 2002

Related U.S. Application Data
(60) Provisional application No. 60/281,716, filed on Apr. 6, 2001.

(51) Int. Cl. C12P 7/64

(52) U.S. Cl. 435/134

(57) ABSTRACT

The present invention is directed to improving productivity of an enzymatic method for producing transesterified fats. Specifically, a method that can greatly improve the productivity of enzymatic transesterification or esterification by purifying the substrate oil to extend the useful life of the enzyme is disclosed. One example of the purification medium is packed silica gel.

### CROSS REFERENCES TO RELATED APPLICATIONS

This application claims the benefit of priority to U.S. Provisional Application No. 60/281,716; filed Apr. 6, 2001, the contents of which are fully incorporated by reference herein.

### BACKGROUND OF THE INVENTION

1. Field of the Invention

The invention relates to methods for producing fats and oils. Specifically, the invention pertains to prolonging the enzymatic activity of lipase used for transesterification or esterification of glycerides, free fatty acids, monohydroxyl alchols, polyhydroxyl alcohols, and esters in the production of fats and oils.

2. Related Art

Fats and oils are composed of triglycerides made up of a glycerol moiety in which the hydroxyl groups are esterified with carboxylic acids. Whereas solid fats tend to be formed by triglycerides having saturated fatty acids, triglycerides with unsaturated fatty acids tend to be liquid (oils) at room temperature. Monoglycerides and diglycerides, having respectively one fatty acid ester and two alcoholic groups or two fatty acid esters and one alcoholic group, are also found in fats and oils as minor components.

Many fats and oils are readily obtained from processing plant or animal matter. However, some fats and oils are obtained via well-known chemical or enzymatic transesterification or esterification processes. By these processes, one or more of the fatty acyl groups on a glyceride is transferred, hydrolyzed or replaced with a different fatty acyl group. Chemical methods require harsh alkaline conditions, high temperatures and generate wasteful by-products. The discolored fats and oils produced need to be neutralized, washed and centrifuged to remove catalysts, and ultimately bleached. In addition to these problems, chemical transesterification or chemical esterification is non-specific in the glyceride position or type of fatty acids transferred, hydrolyzed or replaced. It is thus very difficult or impossible to chemically produce specific fats or oils. In contrast, enzymatic methods of transesterification or esterification are simpler, cleaner, environmentally friendly and are highly specific with respect to modifying glyceride fatty acyl groups.

The enzymes capable of affecting this transesterification or esterification in glycerides are known as lipases. Lipases are obtained from prokaryotic or eukaryotic microorganisms and typically fall into one of three categories (Macrae, A. R., J.A.O.C.S.60:243A-246A (1983)).

The first category includes nonspecific lipases capable of releasing or binding any fatty acid from or to any glyceride position. These lipases provide little benefit over chemical processes. Such lipases have been obtained from Candida cylindracae, Corynebacterium acnes and Staphylococcus aureus (Macrae, 1983; U.S. Pat. No. 5,128,251). The second category of lipases only adds or removes specific fatty acids to or from specific glycerides. Thus, these lipases are only useful in producing or modifying specific glycerides. Such lipases have been obtained from Geotrichum candidium and Rhizopus, Aspergilus, and Mucor genera (Macrae, 1983; U.S. Pat. No. 5,128,251). The last category of lipases catalyze the removal or addition of fatty acids from the glyceride carbons on the end in the 1- and 3-positions. Such lipases have been obtained from Thermomyces lanuginosa, Rhizomucor miehei, Aspergillus niger, Mucor javanicus, Rhizopus delemar, and Rhizopus arrhizus (Macrae, 1983).

The last category of enzymes have wide applicability. For example, cocoa butter consists primarily (about 70-80% by weight) of saturated-oleic-saturated (SOS) triglycerides (EP 0188122 A1). It is this triglyceride composition which provides the unique characteristics by which chocolate products hold their shape at room temperature but melt slightly below human body temperature (see U.S. Pat. No. 4,276,322). These SOS triglycerides include 1,3-dipalmitoyl-2-monooleine (POP), 1(3)-palmitoyl-3(1)-stearoyl-2-monooleine (POSt) and 1,3-distearoyl-2-monooleine (StOSt). Thus, oleic acid-rich glycerides with an oleic ester group in the middle position can be incubated with palmitic and stearic acid in the presence of a 1,3-specific lipase to produce POP, POSt and StOSt, i.e., cocoa butter substitutes (U.S. Pat. No. 4,276,322). The production of cocoa butter substitutes alleviates food manufacturers from widely fluctuating cocoa butter supply and cost.

1,3-specific lipases also are useful in the manufacture of specialty 1,3-diglycerides, as described in U.S. Pat. No. 6,004,611.

Despite these benefits, enzymatic transesterification or esterification is a costly process because of the expense in providing a large amount of purified lipase. Moreover, the enzymatic activity of lipase decays with time and exposure to large amounts of fats or oils. The present invention reduces these problems by providing a method by which the enzymatic activity of lipase is prolonged.

### SUMMARY OF THE INVENTION

The present invention relates to a method for producing fats or oils comprising forming an initial substrate comprising one compound or a mixture of compounds selected from the group consisting of one or more glycerides, free fatty acids, monohydroxyl alchols, polyhydroxyl alcohols, and esters; contacting the initial substrate with one or more types of purification media to generate a purified substrate; contacting the purified substrate with lipase to effect esterification or transesterification creating the fats or oils; wherein lipase enzymatic activity is prolonged.

In one embodiment of the present invention, the initial substrate comprises glycerides selected from the group consisting of butterfat, cocoa butter, cocoa butter substitutes, illipe fat, kokum butter, milk fat, mowrah fat, phulwara butter, sal fat, shea fat, borneo tallow, lard, lanolin, beef tallow, mutton tallow, tallow or other animal fat, canola oil, castor oil, coconut oil, coriander oil, corn oil, cottonseed oil, hazlenut oil, hempseed oil, linseed oil, mango kernel oil, meadowfoam oil, neat's foot oil, olive oil, palm oil, palm kernel oil, peanut oil, rapeseed oil, rice bran oil, safflower oil, sasanqua oil, soybean oil, sunflower seed oil, tall oil, tsubaki oil, vegetable oils, marine oils which can be converted into plastic or solid fats such as menhaden, candlefish oil, cod-liver oil, orange roughy oil, pile herd, sardine oil, whale and herring oils, 1,3-dipalmitoyl-2-monooleine (POP), 1(3)-palmitoyl-3(1)-stearoyl-2-monooleine (POSt), 1,3-distearoyl-2-monooleine (StOSt), glycerol, triglyceride, diglyceride, monoglyceride, behenic acid triglyceride, trioleine, tripalmitine, tristearine, palm olein, palm stearin, palm kernel olein, palm kernel stearin and triglycerides of medium chain fatty acids; or, processed partial or fully hydrogenated or fractionated oils thereof.

In another embodiment of the present invention, the initial substrate comprises esters. Preferably, the esters are selected from the group consisting of wax esters, alkyl esters, methyl esters, ethyl esters, isopropyl esters, octadecyl esters, aryl esters, propylene glycol esters, ethylene glycol esters, 1,2-propanediol esters and 1,3-propanediol esters. Also preferably, the esters are formed from the esterification or transesterification of monohydroxyl alcohols or polyhydroxyl alcohols. Preferably, the monohydroxyl alcohols or the polyhydroxyl alcohols are primary, secondary or tertiary alcohols of annular, straight or branched chain compounds. Also preferably, the monohydroxyl alcohols are selected from the group consisting of methyl alcohol, isopropyl alcohol, ally alcohol, ethanol, propanol, n-butanol, iso-butanol, sec-butanol, tert-butanol, n-pentanol, iso-pentanol, n-hexanol or octadecyl alcohol. Also preferably, the polyhydroxyl alcohols are selected from the group consisting of glycerol, propylene glycol, ethylene glycol, 1,2-propanediol and 1,3-propanediol.

In another embodiment of the present invention, the initial substrate comprises primary, secondary or tertiary monohydroxyl alcohols of annular, straight or branched chain compounds. Preferably, the monohydroxyl alcohols are selected from the group consisting of methyl alcohol, isopropyl alcohol, ally alcohol, ethanol, propanol, n-butanol, iso-butanol, sec-butanol, tert-butanol, n-pentanol, iso-pentanol, n-hexanol or octadecyl alcohol.

In another embodiment of the present invention, the initial substrate comprises primary, secondary or tertiary polyhydroxyl alcohols of annular, straight or branched chain compounds. Preferably, the polyhydroxyl alcohols are selected from the group consisting of glycerol, propylene glycol, ethylene glycol, 1,2-propanediol and 1,3-propanediol.

In another embodiment of the present invention, the initial substrate comprises one or more fatty acids; wherein the one or more fatty acids are saturated, unsaturated or polyunsaturated. Preferably, the one or more fatty acids comprise carbon chains from 4 to 22 carbons long. Also preferably, the fatty acids are selected from the group consisting of palmitic acid, stearic acid, oleic acid, linoleic acid, linolenic acid, arachidonic acid, erucic acid, caproic acid, caprylic acid, capric acid, lauric acid, myristic acid, eicosapentaenoic acid (EPA), docosahexaenoic acid (DHA), 5-eicosenoic acid, butyric acid, γ-linolenic acid and conjugated linoleic acid. Also preferably, the one or more fatty acids comprise carbon chains from 6 to 22 carbons long.

In another embodiment of the present invention, one or more types of purification media and the lipase are packed in one or more columns. Preferably, the columns are jacketed columns in which the temperature of the initial substrate, the purified substrate, the one or more types of purification media or the lipase is regulated.

In another embodiment of the present invention, the purified substrate is prepared by mixing the initial substrate with the one or more types of purification media in a tank for a batch slurry purification reaction or mixing the initial substrate in a series of tanks for a series of batch slurry purification reactions. Preferably, the purified substrate is separated from the one or more types of purification media via filtration, centrifugation or concentration prior to reacting the purified substrate with the lipase. Also preferably, the method of the present invention further comprises mixing the purified substrate with the lipase in a tank for a batch slurry reaction, or flowing the purified substrate through a column containing the lipase.

In another embodiment of the present invention, a bed of the one or more types of purification media is placed upon a bed of the lipase within a column. Preferably, the column is a jacketed column in which the temperature of the initial substrate, the purified substrate, the one or more types of purification media or the lipase is regulated.

In another embodiment of the present invention, the lipase is obtained from a cultured eukaryotic or prokaryotic cell line.

In another embodiment of the present invention, the lipase is a 1,3-selective lipase.

In another embodiment of the present invention, the lipase is a non-selective lipase.

In another embodiment of the present invention, the purification medium is selected from the group consisting of activated carbon, coal activated carbon, wood activated carbon, peat activated carbon, coconut shell activated carbon, natural minerals, processed minerals, montmorillonite, attapulgite, bentonite, palygorskite, Fuller's earth, diatomite, smectite, hormite, quartz sand, limestone, kaolin, ball clay, talc, pyrophyllite, perlite, silica, sodium silicate, silica hydrogel, silica gel, fumed silica, precipitated silica, dialytic silica, fibrous materials, cellulose, cellulose esters, cellulose ethers, microcrystalline cellulose; alumina, zeolite, starches, molecular sieves, previously used immobilized lipase, diatomaceous earth, ion exchange resin, size exclusion chromatography resin, chelating resins, chiral resins, rice hull ash, reverse phase silica, and bleaching clays.

In another embodiment of the present invention, the purification medium is silica having a surface area from 200 to 750 m²/g, a mesh value from 3 to 425, an average particle size from 4-200 μ, an average pore radius from 20 to 150 Å, and an average pore volume from 0.68 to 1.15 cm³/g. Preferably the silica is 35-60 mesh with an average pore size of about 60 Å.

In another embodiment of the present invention, the method further comprises (a) monitoring enzymatic activity by measuring one or more physical properties of the fats or oils after having contacted the lipase; (b) adjusting the duration of time for which the purified substrate contacts the lipase, or adjusting the temperature of the initial substrate, the purified substrate, the one or more types of purification media or the lipase; and (c) adjusting the amount and type of the one or more types of purification media in response to changes in the physical properties to optimize the enzymatic activity. Preferably the one or more physical properties include the Mettler dropping point temperature of the fats or oils. Also preferably the one or more physical properties include the solid fat content temperature profile of the fats or oils.

In another embodiment of the present invention, the fats or oils produced are 1,3-diglycerides.

### BRIEF DESCRIPTION OF THE FIGURES

FIG. 1 is a graph showing the decay of lipase enzymatic activity as measured by the decrease in product flow rate where a piston pump is used without purification medium (closed diamonds), where a peristaltic pump is used without purification medium (open squares), and where a piston pump is used with purification medium (open triangles).

### DETAILED DESCRIPTION OF THE PREFERRED EMBODIMENT

For purposes herein, the term substrate refers to one or any combination of the following materials: glycerides, triglycerides, diglycerides, monoglycerides, free fatty acids, monohydroxyl alcohols, polyhydroxyl alcohols or esters. The term initial substrate refers to a substrate for which the process of purification by contacting the initial substrate with one or more purification media has not yet been completed. The term purified substrate refers to a substrate that has been purified and is ready to contact lipase. The term product is used interchangeably with esterified or transesterified fats, oils, glycerides, triglycerides, diglycerides, monoglycerides, free fatty acids, monohydroxyl alcohols, polyhydroxyl alcohols or esters created or produced via the enzymatic transesterification or esterification activity of the lipase. Product also refers to a fluid or solid at room temperature increased in its proportional content of transesterified fats, oils, glycerides, triglycerides, diglycerides, monoglycerides, free fatty acids, monohydroxyl alcohols, polyhydroxyl alcohols or esters as a result of its having contacted lipase. Transesterified or esterified product is to be distinguished from the contents of initial substrate or purified substrate in that product has undergone additional enzymatic transesterification or esterification reaction. The contents of initial substrate or purified substrate could have already undergone none, or one or more enzymatic transesterification or esterification reactions. The term fatty acid is used interchangeably with the term free fatty acid or fatty acyl group.

The present invention relates to a method for producing fats or oils comprising forming an initial substrate comprising one compound or a mixture of compounds selected from the group consisting of one or more glycerides, free fatty acids, monohydroxyl alchols, polyhydroxyl alcohols, and esters; contacting the initial substrate with one or more types of purification media to generate a purified substrate; contacting the purified substrate with lipase to effect esterification or transesterification creating the fats or oils; wherein lipase enzymatic activity is prolonged.

Also preferably, the present invention relates to a method for producing fats or oils comprising forming an initial substrate comprising one compound or a mixture of compounds selected from the group consisting of one or more glycerides, free fatty acids, monohydroxyl alchols, polyhydroxyl alcohols, and esters; contacting the initial substrate with one or more types of purification media to generate a purified substrate comprising the compound proportionally enhanced in content relative to its content in the initial substrate; contacting the purified substrate with lipase to effect esterification or transesterification creating the product; wherein lipase enzymatic activity is prolonged.

Esterification or transesterification are the processes by which an acyl group is added, hydrolyzed, repositioned or replaced on a glyceride, monoglyceride, diglyceride, triglyceride, monohydroxyl alcohol, polyhydroxyl alcohol, ester, or free fatty acid. The acyl group can be derived from a monoglyceride, diglyceride, triglyceride, ester, or free fatty acid. The alkyl moiety of the acyl group can be straight or branched, saturated or unsaturated, or contain non-carbon substituents including oxygen, sulfur or nitrogen.

Transesterification or esterification is affected by a lipase, which is preferably obtained from a cultured eukaryotic or prokaryotic cell line. The lipase can be unspecific or specific with respect to its substrate. Preferably, the lipase is a 1,3-selective lipase, which catalyzes transesterification of the terminal esters in the 1 and 3 positions of a glyceride. The lipase can also preferably be a non-selective, nonspecific lipase.

The initial substrate can be composed of one type of glyceride fat or oil and have its physical properties modified in a process known as randomization. For example, when fully hydrogenated palm kernel oil is treated with lipase capable of randomization, the components of the product have different physical properties. Both 1,3-selective lipases and nonselective lipases such as Candida cylindracae lipase are capable of this randomizing process.

There are many microorganisms from which lipases useful in the present invention are obtained. U.S. Pat. No. 5,219,733 lists examples of such microorganisms including those of the genus Achromobacter such as A. iofurgus and A. lipolyticum, the genus Chromobacterium such as C. viscosum var. paralipolyticum; the genus Corynebacterium such as C. acnes; the genus Staphylococcus such as S. aureus; the genus Aspergillus such as A. niger and A. oryzae; the genus Candida such as C. cylindracea, C. antarctica b, C. rosa and C. rugosa; the genus Humicora such as H. lanuginosa; the genus Penicillium such as P. caseicolum, P. crustosum, P. cyclopium and P. roqueforti; the genus Torulopsis such as T. ernobii; the genus Mucor such as M. miehei, M. japonicus and M. javanicus; the genus Bacillus such as B. subtilis; the genus Thermomyces such as T. ibadanensis and T. lanuginosa (see Zhang, H. et al. J.A.O.C.S. 78: 57-64 (2001)); the genus Rhizopus such as R. delemar, R. japonicus, R. arrhizus and R. neveus; the genus Pseudomonas such as P. aeruginosa, P. fragi, P. cepacia, P. mephitica var. lipolytica and P. fluorescens; the genus Alcaligenes; the genus Rhizomucor such as R. miehei; the genus Humicolo such as H. rosa; and the genus Geotrichum such as G. candidum. Several lipases obtained from these organisms are commercially available as purified enzymes.

Lipases obtained from the organisms above are immobilized for the present invention using suitable carriers by a usual method known to persons of ordinary skill in the art. U.S. Pat. Nos. 4,798,793; 5,166,064; 5,219,733; 5,292,649; and 5,773,266 describe examples of immobilized lipase and methods of preparation. Examples of methods of preparation include the entrapping method, inorganic carrier covalent bond method, organic carrier covalent bond method, and the adsorption method. The lipase used in the examples below were obtained from Novozymes (Denmark) but can be substituted with purified and/or immobilized lipase prepared by others. The present invention also contemplates using crude enzyme preparations or cells of microorganisms capable of overexpressing lipase, a culture of such cells, a substrate enzyme solution obtained by treating the culture, or a composition containing the enzyme.

U.S. Pat. Nos. 4,940,845 and 5,219,733 describe the characteristics of several useful carriers. Useful carriers are preferably microporous and have a hydrophobic porous surface. Usually, the pores have an average radius of about 10 Å to about 1,000 Å, and a porosity from about 20 to about 80% by volume, more preferably, from about 40 to about 60% by volume. The pores give the carrier an increased enzyme bonding area per particle of the carrier. Examples of preferred inorganic carriers include porous glass, porous ceramics, celite, porous metallic particles such as titanium oxide, stainless steel or alumina, porous silica gel, molecular sieve, active carbon, clay, kaolinite, perlite, glass fibers, diatomaceous earth, bentonite, hydroxyapatite, calcium phosphate gel, and alkylamine derivatives of inorganic carriers. Examples of preferred organic carriers include microporous Teflon, aliphatic olefinic polymer (e.g., polyethylene, polypropylene, a homo- or copolymer of styrene or a blend thereof or a pretreated inorganic support) nylon, polyamides, polycarbonates, nitrocellulose and acetylcellulose. Other suitable organic carriers include hydrophillic polysaccharides such as agarose gel with an alkyl, phenyl, trityl or other similar hydrophobic group to provide a hydrophobic porous surface (e.g., “Octyl-Sepharose CL-4B”, “Phenyl-Sepharose CL-4B”, both products of Pharmacia Fine Chemicals). Microporous adsorbing resins include those made of styrene or alkylamine polymer, chelate resin, ion exchange resin such a “DOWEX MWA-1” (weakly basic anion exchange resin manufactured by the Dow Chemical Co., having a tertiary amine as the exchange group, composed basically of polystyrene chains cross linked with divinylbenzene, 150 Å in average pore radius and 20-50 mesh in particle size), and hydrophilic cellulose resin such as one prepared by masking the hydrophilic group of a cellulosic carrier, e.g., “Cellulofine GC700-m” (product of Chisso Corporation, 45-105 μm in particle size).

In the method of the present invention, a free fatty acid is a carboxylic acid with a carbon chain up to 40 carbons long. The free fatty acids are saturated, unsaturated or polyunsaturated.

Examples of fatty acids useful in the present invention include saturated straight-chain or branched fatty acids, unsaturated straight-chain or branched fatty acids, hydroxy fatty acids, and polycarboxylic acids. The fatty acids can be naturally occurring, processed or refined from natural products or synthetically produced. Although there is no upper or lower limit for the length of the longest carbon chain in useful fatty acids, it is preferable that their length is about 6 to about 34 carbons long. Specific fatty acids useful for the present invention are described in U.S. Pat. Nos. 4,883,684; 5,124,166;

5,149,642; 5,219,733; 5,399,728.

Examples of useful saturated straight-chain fatty acids having an even number of carbon atoms described in U.S. Pat. No. 5,219,733 include acetic acid, butyric acid, caproic acid, caprylic acid, capric acid, lauric acid, myristic acid, palmitic acid, stearic acid, arachic acid, behenic acid, lignoceric acid, hexacosanoic acid, octacosanoic acid, triacontanoic acid and n-dotriacontanoic acid, and those having an odd number of carbon atoms, such as propionic acid, n-valeric acid, enanthic acid, pelargonic acid, hendecanoic acid, tridecanoic acid, pentadecanoic acid, heptadecanoic acid, nonadecanoic acid, heneicosanoic acid, tricosanoic acid, pentacosanoic acid and heptacosanoic acid.

Examples of useful saturated branched fatty acids described in U.S. Pat. No. 5,219,733 include isobutyric acid, isocaproic acid, isocaprylic acid, isocapric acid, isolauric acid, 11-methyldodecanoic acid, isomyristic acid, 13-methyl-tetradecanoic acid, isopalmitic acid, 15-methyl-hexadecanoic acid, isostearic acid, 17-methyloctadecanoic acid, isoarachic acid, 19-methyl-eicosanoic acid, a-ethyl-hexanoic acid, a-hexyldecanoic acid, a-heptylundecanoic acid, 2-decyltetradecanoic acid, 2-undecyltetradecanoic acid, 2-decylpentadecanoic acid, 2-undecylpentadecanoic acid, and Fine oxocol 1800 acid (product of Nissan Chemical Industries, Ltd.)

Examples of useful saturated odd-carbon branched fatty acids described in U.S. Pat. No. 5,219,733 include anteiso fatty acids terminating with an isobutyl group, such as 6-methyl-octanoic acid, 8-methyl-decanoic acid, 10-methyl-dodecanoic acid, 12-methyl-tetradecanoic acid, 14-methyl-hexadecanoic acid, 16-methyl-octadecanoic acid, 18-methyl-eicosanoic acid, 20-methyl-docosanoic acid, 22-methyl-tetracosanoic acid, 24-methyl-hexacosanoic acid and 26-methyloctacosanoic acid.

Examples of useful unsaturated fatty acids described in U.S. Pat. No. 5,219,733 include 4-decenoic acid, caproleic acid, 4-dodecenoic acid, 5-dodecenoic acid, lauroleic acid, 4-tetradecenoic acid, 5-tetradecenoic acid, 9-tetradecenoic acid, palmitoleic acid, 6-octadecenoic acid, oleic acid, 9-octadecenoic acid, 11-octadecenoic acid, 9-eicosenoic acid, cis-11-eicosenoic acid, cetoleic acid, 13-docosenoic acid, 15-tetracosenoic acid, 17-hexacosenoic acid, 6,9,12,15-hexadecatetraenoic acid, linoleic acid, linolenic acid, a-eleostearic acid, b-eleostearic acid, punicic acid, 6,9,12,15-octadecatetraenoic acid, parinaric acid, 5,8,11,14-eicosatetraenoic acid, 5,8,11,14,17-eicosapentaenoic acid (EPA), 7,10,13,16,19-docosapentaenoic acid, 4,7,10,13,16,19-docosahexaenoic acid (DHA) and the like.

Examples of useful hydroxy fatty acids described in U.S. Pat. No. 5,219,733 include α-hydroxylauric acid, α-hydroxymyristic acid, α-hydroxypalmitic acid, α-hydroxystearic acid, ω-hydroxylauric acid, α-hydroxyarachic acid, 9-hydroxy-12-octadecenoic acid, ricinoleic acid, α-hydroxybehenic acid, 9-hydroxy-trans-10,12-octadecadienic acid, kamolenic acid, ipurolic acid, 9,10-dihydroxystearic acid, 12-hydroxystearic acid and the like.

Examples of useful polycarboxylic acids described in U.S. Pat. No. 5,219,733 include oxalic acid, citric acid, malonic acid, succinic acid, glutaric acid, adipic acid, pimelic acid, suberic acid, azelaic acid, sebacic acid, D,L-malic acid and the like.

For the reaction of the present invention, these fatty acids can be used singly, or at least two of such acids of the same group or different groups are usable in admixture. Preferably, the free fatty acids have carbon chains from 4 to 34 carbons long. More preferably, the free fatty acids have carbon chains from 4 to 26 carbons long. Most preferably, the free fatty acids have carbon chains from 4 to 22 carbons long. Preferably the free fatty acids are selected from the following group: palmitic acid, stearic acid, oleic acid, linoleic acid, linolenic acid, arachidonic acid, erucic acid, caproic acid, caprylic acid, capric acid, eicosapentanoic acid (EPA), docosahexaenoic acid (DHA), lauric acid, myristic acid, 5-eicosenoic acid, butyric acid, γ-linolenic acid and conjugated linoleic acid. Fatty acids derived from various plant and animal fats and oils (such as fish oil fatty acids) and processed or refined fatty acids from plant and animal fats and oils (such as fractionated fish oil fatty acids in which EPA and DHA are concentrated) can also be added. Medium chain fatty acids (as described by Merolli, A. et al., INFORM, 8:597-603 (1997)) can also be used. Also preferably, the free fatty acids have carbon chains from 6 to 36, 6 to 24 or 6 to 22 carbons long.

Glycerides useful in the present invention include molecules given by the chemical formula CH₂RCHR′CH₂R″ wherein R, R′ and R″ are alcohols (OH) or acyl fatty acids given by OC(O)R′″ wherein R′ is a saturated, unsaturated or polyunsaturated, straight or branched carbon chain up to 40 carbons long. R, R′ and R″ can be the same or different. The esters R, R′ and R″ can be obtained from any of the fatty acids described herein. Glycerides for the present invention include triglycerides in which R, R′ and R″ are all acyl groups, diglycerides in which two of R, R′ and R″ are acyl groups and one alcohol functionality is present, monoglycerides in which only one of R, R′ and R″ is an acyl group and two alcoholic functionalities are present, or glycerol. Glycerides useful as starting materials of the invention include natural, processed, refined and synthetic fats and oils. Refined fats and oils are described in Stauffer, C., Fats and Oils, Eagan Press, St. Paul, Minn. Examples of processed fats and oils are hydrogenated and fractionated fats and oils.

Glycerides for the method of the present invention are selected from the following: butterfat, cocoa butter, cocoa butter substitutes, illipe fat, kokum butter, milk fat, mowrah fat, phulwara butter, sal fat, shea fat, borneo tallow, lard, lanolin, beef tallow, mutton tallow, tallow or other animal fat, canola oil, castor oil, coconut oil, coriander oil, corn oil, cottonseed oil, hazlenut oil, hempseed oil, linseed oil, mango kernel oil, meadowfoam oil, neat's foot oil, olive oil, palm oil, palm kernel oil, peanut oil, rapeseed oil, rice bran oil, safflower oil, sasanqua oil, soybean oil, sunflower seed oil, tall oil, tsubaki oil, vegetable oils, marine oils which can be converted into plastic or solid fats such as menhaden, candlefish oil, cod-liver oil, orange roughy oil, pile herd, sardine oil, whale and herring oils, 1,3-dipalmitoyl-2-monooleine (POP), 1(3)-palmitoyl-3(1)-stearoyl-2-monooleine (POSt), 1,3-distearoyl-2-monooleine (StOSt), glycerol, triglyceride, diglyceride, monoglyceride, behenic acid triglyceride, trioleine, tripalmitine, tristearine and triglycerides of medium chain fatty acids. Processed fats and oils such as hydrogenated or fractionated fats and oils can also be used. Examples of fractionated fats include palm olein, palm stearin, palm kernel olein, and palm kernel stearin. Either fully hydrogenated or partially hydrogenated oils of the above are also useful.

Hydrogenated or unsaturated forms of the above listed oils are also useful for the present invention. For the method of this invention, these fatty acid esters are usable singly, or at least two of them can be used in admixture. Also, one or more of these esters can be used with one or more fatty acids.

Examples of alcohols useful in the present invention include monohydroxyl alcohols or polyhydroxyl alcohols. The monohydroxyl alcohols can be primary, secondary or tertiary alcohols of annular, straight or branched chain compounds with one or more carbons such as methyl alcohol, isopropyl alcohol, ally alcohol, ethanol, propanol, n-butanol, iso-butanol, sec-butanol, tert-butanol, n-pentanol, iso-pentanol, n-hexanol or octadecyl alcohol. The hydroxyl group can be attached to an aromatic ring, such as phenol. Examples of polyhydroxyl alcohols includes glycerol, propylene glycol, ethylene glycol, 1,2-propanediol and 1,3-propanediol.

U.S. Pat. No. 5,219,733 indicates other alcohols useful for the present invention. These alcohols include, but are not limited to 14-methylhexadecanol-1, 16-methyloctadecanol-1, 18-methylnonadecanol, 18-methyleicosanol, 20-methylheneicosanol, 20-methyldocosanol, 22-methyltricosanol, 22-methyltetracosanol, 24-methylpentacosanol-1 and 24-methyleicosanol.

The initial substrate can comprise esters. Examples of useful esters other than glycerides include wax esters, alkyl esters such as methyl, ethyl, isopropyl or octadecyl esters, aryl esters, propylene glycol esters, ethylene glycol esters, 1,2-propanediol esters and 1,3-propanediol esters. Esters can be formed from the esterification or transesterification of monohydroxyl alcohols or polyhydroxyl alcohols.

The present invention can be used in batch slurry type reactions as described in Example 4, in which the slurry of lipases and substrates are mixed vigorously to ensure a good contact between them. Preferably, the transesterification or esterification reaction is carried out in a fixed bed reactor with immobilized lipases.

Fats and oils undergo degradation in part because of the presence of minor impurities. These impurities, as well as degradation products such as oxidized fats and oils, can detrimentally affect lipase enzymatic activity. Other oxidative species include agents that initiate self-propagated radical reaction pathways, oxygen or other reactive oxygen species (such as peroxides, ozone, superoxide, etc.) that are capable of oxidizing fats, oils or enzymes. However, these detrimental effects are not fully understood. The present invention discloses a method that greatly improves the productivity of enzymatic transesterification or esterification by purifying the substrate oil to extend the useful life of the enzyme. The examples described below show that productivity of the enzymatic transesterification or esterification is improved greatly by purification of the substrate oil. One example of the purification means is silica gel packed in a column for pre-column purification of the substrate. However, it is also contemplated that the silica gel can be provided as a packed bed on top of the packed lipase.

The purification medium of the present invention is preferably silica having a surface area from 200 to 750 m²/g, a mesh value from 3 to 425, an average particle size from 4-200 μ, an average pore radius from 20 to 150 Å, and an average pore volume from 0.68 to 1.15 cm³/g. Also preferably the silica gel is 35-60 mesh with an average pore size of 60 Å.

It is also contemplated that the purification medium useful in the present invention can be selected from one of the following: activated carbon, coal activated carbon, wood activated carbon, peat activated carbon, coconut shell activated carbon, natural minerals, processed minerals, montmorillonite, attapulgite, bentonite, palygorskite, Fuller's earth, diatomaceous earth, diatomite, smectite, hormite, quartz sand, limestone, kaolin, clays, ball clay, talc, pyrophyllite, perlite, silica, sodium silicate, silica hydrogel, silica gel, fumed silica, precipitated silica, dialytic silica, TriSyl® silica, fibrous materials, cellulose, cellulose esters, cellulose ethers, microcrystalline cellulose, Avicel®, alumina, zeolite, starches, molecular sieves, previously used immobilized lipase, ion exchange resin, size exclusion chromatography resin, chelating resins, chiral resins, rice hull ash, reverse phase silica, and bleaching clays. The purification medium can be resinous, granulated, particulate, membranous or fibrous.

In the method of the present invention, one or more types of purification media and the lipase are packed into one or more columns. If multiple types of purification media are used, they can be mixed together and packed into a single column or kept separate in different columns. In an alternative embodiment, one or more types of purification media are placed upon a bed of packed lipase within a column. Alternatively, the lipase can be kept separate from the purification media by packing it in its own column. More than one type of purification media can be used for purposes of removing different kinds of impurities in the initial substrate. The columns and other fluid conduits can be jacketed so as to regulate the temperature of the initial substrate, the purified substrate, the purification media or the lipase. The purification media can be regenerated for repeated use.

Also in the method of the present invention, the purified substrate is prepared by mixing the initial substrate with one or more types of purification media in a tank for a batch slurry type purification reaction or mixing the initial substrate in a series of tanks for a series of batch slurry type purification reactions. In these batch slurry type purification reactions, the different types of purification media can be kept separate or can be combined. After reacting with one type of purification medium (or specific mixture of purification media), the initial substrate is separated from the purification medium (or media) via filtration, centrifugation or concentration. After this separation step, the initial substrate is further purified with other purification media or serves as purified substrate and is reacted with lipase. The reaction of purified substrate prepared by this batch slurry type purification reaction method can be reacted with lipase in a tank for batch slurry type transesterification or esterification. Alternatively, the purified substrate can be caused to flow through a lipase column. The reacting tanks, columns and other fluid conduits can be jacketed so as to regulate the temperature of the initial substrate, the purified substrate, the purification media or the lipase. Other manners of temperature regulation, such as heating/cooling coils or temperature controlled rooms, are contemplated and well known in the art. The purification media can be regenerated for repeated use.

Lipase enzymatic activity is also affected by factors such as temperature, light and moisture content. Temperature is controlled as described above. Light can be kept out by using various light blocking or filtering means known in the art. Moisture content, which includes ambient atmospheric moisture, is controlled by operating the process as a closed system. The closed system can be under a positive nitrogenous pressure to expel moisture. Alternatively, a bed of nitrogen gas can be placed on top of the substrate, purification bed or column, or packed lipase column. Other inert gasses such as helium or argon can also be used. These techniques have the added benefit of keeping atmospheric oxidative species (including oxygen) away from the substrate, product or enzyme.

Resinous immobilized lipase can be mixed with initial or purified substrate to form a slurry which is packed into a suitable column. Initial substrate is prepared from one or more glycerides, monoglycerides, diglycerides, triglycerides, free fatty acids, monohydroxyl alchols, polyhydroxyl alcohols and esters. The temperature of the substrate is regulated so that it can continuously flow though the column for contact with the lipase and transesterification or esterification. If solid glycerides or fatty acids are used, the substrate is heated to a fluid state. The substrate can be caused to flow through the column(s) under the force of gravity, by using a peristaltic or piston pump, under the influence of a suction or vacuum pump, or using a centrifugal pump. The transesterified fats and oils produced are collected and the desired glycerides are separated from the mixture of reaction products by methods well known in the art. This continuous method involves a reduced likelihood of permitting exposure of the substrates to air during reaction and therefore has the advantage that unsaturated fatty acids, glycerides or the like, if used, will not be exposed to moisture or oxidative species. Alternatively, reaction tanks for batch slurry type production as described above can also be used. Preferably, these reaction tanks are also sealed from air so as to prevent exposure to oxygen, moisture, or other ambient oxidizing species.

The method of the present invention comprises monitoring enzymatic activity by measuring one or more physical properties of the fats or oils after having contacted the lipase; adjusting the duration of time for which the purified substrate contacts the lipase; and adjusting the amount and type of the one or more types of purification media in response to changes in said physical properties to optimize said enzymatic activity.

The method of the present invention also comprises monitoring enzymatic activity by measuring one or more physical properties of the fats or oils after having contacted the lipase; adjusting the temperature of the initial substrate, the purified substrate, the one or more types of purification media or the lipase; and adjusting the amount and type of the one or more types of purification media in response to changes in said physical properties to optimize said enzymatic activity.

In the present invention, changes in lipase enzymatic activity can be followed by monitoring the transesterified fats and oils which have flowed through the packed lipase. The substrate and product have different characteristic physical properties which are used to determine the lipase activity. For example, the Mettler dropping point (MDP, American Oil Chemists Society Official Method #Cc 18-80) is a technique well known in the art for measuring the temperature at which a mixture of fats or oils becomes fluid. The product's solid fat content (SFC) profile at different temperatures can also be measured (American Oil Chemists Society Official Method #Cd 16b-93).

Where purified substrate is passed though a lipase column, enzymatic activity can be measured by reducing the flow rate of the substrate in response to changes in the product's MDP temperature. The substrate and product each have a characteristic MDP temperature. As the lipase enzymatic activity decays, less substrate is converted into product resulting in an increased substrate:product ratio. This increased ratio results in a change of MP temperature of the outflowing fats or oils tending towards the MDP temperature value of the non-transesterified substrate. To minimize this change, the flow rate of the substrate is reduced so that it is exposed for a longer period of time to the packed lipase. The flow rate reduction increases the product:substrate ratio and consequently the MDP temperature of the outflowing fats or oils or glycerides reflect that of transesterified product. However, a reduced flow rate generates a reduced quantity of product. The flow rate is iteratively reduced until the product possesses the targeted MDP. The reduction in flow rate can be correlated with reduction of desired glyceride product, which can be correlated to changes in enzymatic activity. Thus, monitoring and maintaining the MDP temperature is useful for calculating changes in enzymatic activity.

The SFC temperature profile is also useful for calculating changes in enzymatic activity. The SFC temperature profile is a measure of the solid fat content as a function of temperature. Substrate and product each have characteristic SFC temperature profiles. As the lipase activity decays, the outflowing fats and oils have a change in profile that tends towards that of the substrate. The substrate flow rate is reduced to maintain a desired SFC temperature profile. As described above, this reduction in flow rate is useful for calculating changes in enzymatic activity. Thus, monitoring and maintaining the SFC temperature profile is useful for calculating changes in enzymatic activity.

Enzymatic activity can also be measured by reducing the flow rate of the fat or oil substrate in response to changes in the optical spectroscopic characteristics of the product. The substrate and product each have a characteristic optical spectrum. As the lipase activity decays, the amount of product that gives rise to its characteristic spectroscopic signal diminishes. Again, the flow rate is iteratively reduced until the outflowing product again displays its characteristic spectroscopic signal. The reduction in flow rate can be correlated with reduction of desired glyceride product, which can be correlated to changes in enzymatic activity. Thus, monitoring and maintaining the product's optical spectrum is useful for calculating changes in enzymatic activity. Alternatively, changes in the refractive index of the fat or oil substrate can be monitored.

Where purified substrate is reacted with lipase in a tank for batch slurry type production, changes in the product's physical properties can also be monitored as described above. In a batch slurry type process, an optimized duration of time is determined for contacting the initial substrate with the purification medium (or media). An optimized time is also determined for contacting the purified substrate with lipase.

By examining the product's MDP temperature, SFC temperature profile, optical spectroscopic signals or other physical changes, the lipase activity can be closely monitored. Because some amount of decay in activity is inevitable, substrate flow rate must be reduced with the progression of time.

However, by experimenting with the amount and type of purification medium, an optimized system is arranged wherein the decay of enzymatic activity is reduced.

Thus, the present invention involves monitoring enzymatic activity by measuring one or more physical properties of said fats or oils after having flowed through said lipase, adjusting flow rate, column residence time, or temperature of said substrate mixture or said purified substrate mixture, and adjusting the amount and type of said purification medium in response to changes in said physical properties to optimize said enzymatic activity.

When the initial substrate consists of one or more glyceride oils, the product transesterified oil can be subjected to usual oil refining processes, such as deodorization, to make it desirable as edible oils. When the initial substrate consists of glycerides and free fatty acids, the desired glycerides obtained by the present process can be separated from the reaction mixture by a usual method, such as described in U.S. Pat. No. 5,219,733. In the case of batch slurry type methods, the desired product can be separated using a suitable solvent such as ether, removing the unreacted fatty acid material with an alkali, dehydrating and drying the solvent layer, and removing the solvent from the layer. The desired product can be purified, for example, by column chromatography. Preferably, the method of the present invention produces transesterified or esterified fats with no or reduced trans fatty acids for margarine, shortening, and other confectionery fats such as cocoa butter substitute.

The desired fats or oils thus obtained are usable for a wide variety of culinary applications.

The following examples show the effect of the substrate pretreatment on the enzyme productivity.

### EXAMPLES

The following examples are illustrative only and are not intended to limit the scope of the invention as defined by the appended claims.

In Example 1 and 2, the transesterification was performed without any pretreatment. In both of the examples, a rapid loss of enzyme activity was observed at the beginning of the column operation. Estimated half-lives during this period of rapid activity loss were 6 to 14 days; then, the rate of activity loss slowed, giving half-lives estimations of 28 to 30 days. A rapid loss of activity was observed, again, after running the column for about 30 days. In contrast, Example 3 demonstrates that the operation with a silica purification column did not have an initial period of rapid enzyme activity loss. Rather, the half-life estimation was about 30 days; then, the activity loss even slowed to give about 50-day estimation for the second half-life.

### Example 1

22 g of enzyme (Novozymes' Lipozyme® TL IM) was mixed with liquid soybean oil and packed into a jacketed glass column (2.7-cm diameter) The soy oil was flushed out by pumping the actual substrate (fully hydrogenated soy oil : liquid soy oil=27 :73). The column and the substrate were maintained at 65° C. Extent of enzyme reaction could be monitored very well by the change of melting properties of the substrate and products, which was measured as Mettler droping point (MDP). Oil flow of the column was adjusted so as to have the products' MDP at 117-118° F. Enzyme activity was calculated by comparing the flow rates at which the products have similar MDPs near 117-118° F.

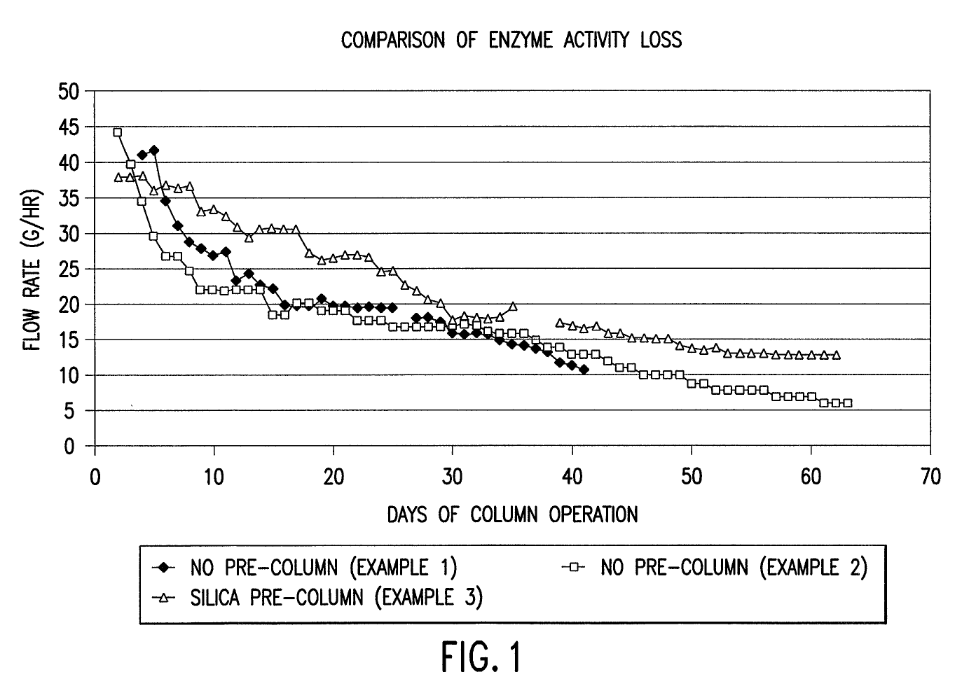

Table 1 summarizes the results. There was a quick activity drop for the first 2 weeks; then the activity drop slowed down. The enzyme activity at Day 13 was about 60% level of that at Day 4. There was another quick activity drop after Day 30. FIG. 1 (closed diamonds) shows the data in greater detail.

TABLE 1
Summary Results of the Column Operation Without Silica
Pretreatment as in Example 1

| | |
|---|---|
| ˜Day 4 | Flushing out soy oil from the column & flow rate |
| | adjustment |
| Day 4 ˜ Day 7 | 25% activity drop in 3 days (6-day half-life |
| | estimation) |
| Day 7 ˜ Day 10 | 13% drop in 3 days (12-day half-life estimation) |
| Day 10 ˜ Day 13 | 11% drop in 3 days (14-day half-life estimation) |
| Day 13 ˜ Day 25 | 20% drop in 12 days (30-day half-life estimation) |
| Day 26 | Total draining of column happened. |
| Day 13 ˜ Day 35 | 40% drop in 22 days (29-day half-life estimation) |
| Day 27 ˜ Day 35 | 20% drop in 8 days (20-day half-life estimation) |
| Day 36 ˜ Day 41 | 25% drop in 5 days (10-day half-life estimation) |

### Example 2

An enzyme column was prepared and run in the same way as described in Example 1, except using a peristaltic pump instead of a piston pump, for replication. Table 2 summarizes the results. As in Example 1, there was a quick activity drop for the first 2 weeks; then, the activity drop slowed down. However, there was another quick activity drop after Day 35. FIG. 1 (open squares) shows the data in greater detail.

TABLE 2
Summary Results of the Column Operation Without Silica
Pretreatment as in Example 2

| | |
|---|---|
| ˜Day 2 | Flushing out soy oil & flow rate adjustment |
| Day 2 ˜ Day 8 | 44% activity drop in 6 days (7-day half-life estimation) |
| Day 2 ˜ Day 12 | 49% drop in 10 days (10-day half-life estimation) |
| Day 12 ˜ Day 35 | 28% drop in 23 days (40-day half-life estimation) |
| Day 35 ˜ Day 46 | 37% drop in 11 days (15-day half-life estimation) |
| Day 45 ˜ Day 51 | 18% drop in 6 days (16-day half-life estimation) |

### Example 3

An enzyme column was prepared as described in Example 1 and 2, and 38 g of silica gel (35-60 mesh, 60 Å) was placed on top of the enzyme bed. Conditions for column operation and analysis were the same as in the previous examples. Table 3 summarizes the results.

There was no quick activity drop in the beginning of the column operation, and the half-life estimation at the time was about 30 days. Even longer half-life estimation was observed as the column was operating for an extended period. FIG. 1 (open triangles) shows the data in greater detail.

TABLE 3
Summary Results of the Column Operation with Silica Pre-
Column Treatment

| | |
|---|---|
| ˜Day 2 | Flushing out soy oil & flow rate adjustment |
| Day 2 ˜ Day 9 | 13% activity drop in 7 days (28-day half-life |
| | estimation) |
| Day 9 ˜ Day 34 | 46% drop in 25 days (27-day half-life estimation) |
| Day 34 ˜ Day 46 | 15% drop in 12 days (41-day half-life estimation) |
| Day 45 ˜ Day 60 | 15% drop in 15 days (50-day half-life estimation) |

### Example 4

400 g of the substrate oil (fully hydrogenated soy oil:corn oil=27:73) in a 1-L flask was heated to 70° C. before adding 40 g of Novozymes' Lipozyme® TL IM lipase. The enzyme/oil slurry was stirred vigorously at the temperature, and samples were taken after 1, 2, 3, 4, 8 and 18 hours of reaction. After the batch reaction, the enzyme was separated from the product oil by filtering the slurry through a filter paper with 2.7-micron particle retention. Table 4 shows the SFC temperature profiles and free fatty acid contents of the samples. The batch reaction yielded more than 10 times greater free fatty acids. The reaction seemed to reach equilibrium after 8 hours of reaction.

TABLE 4
SFC Temperature Profiles and Free Fatty Acid (FFA) Contents of
the Batch Reaction Samples

| SFC | 1 hr | 2 hr | 3 hr | 4 hr | 8 hr | 18 hr | Feed |
|---|---|---|---|---|---|---|---|
| 50° F. | 18.090 | 15.493 | 15.128 | 14.237 | 14.730 | 14.873 | 30.833 |
| 70° F. | 18.297 | 12.905 | 10.739 | 9.130 | 8.387 | 7.816 | 28.032 |
| 80° F. | 17.013 | 12.047 | 9.089 | 7.844 | 6.848 | 6.991 | 26.096 |
| 92° F. | 12.963 | 8.558 | 7.062 | 5.643 | 5.194 | 4.425 | 24.246 |
| 100° F. | 10.318 | 6.711 | 4.307 | 3.433 | 2.831 | 2.562 | 22.215 |
| % FFA | 4.88% | 5.02% | 5.36% | 5.27% | 5.49% | 5.47% | 0.066% |

All publications mentioned herein above are hereby incorporated in their entirety by reference.

While the foregoing invention has been described in some detail for purposes of clarity and understanding, it will be appreciated by one skilled in the art from a reading of this disclosure that various changes in form and detail can be made without departing from the true scope of the invention and appended claims.

We claim:

1. A method for producing fats or oils comprising:
(a) forming an initial substrate comprising one compound or a mixture of compounds selected from the group consisting of one or more glycerides, free fatty acids, monohydroxyl alchols, polyhydroxyl alcohols, and esters;
(b) contacting said initial substrate with one or more types of purification media to generate a purified substrate;
(c) contacting said purified substrate with lipase to effect esterification or transesterification creating said fats or oils;
wherein lipase enzymatic activity is prolonged.

2. The method of claim 1, wherein said initial substrate comprises glycerides and said glycerides are selected from the group consisting of butterfat, cocoa butter, cocoa butter substitutes, illipe fat, kokum butter, milk fat, mowrah fat, phulwara butter, sal fat, shea fat, borneo tallow, lard, lanolin, beef tallow, mutton tallow, tallow or other animal fat, canola oil, castor oil, coconut oil, coriander oil, corn oil, cottonseed oil, hazlenut oil, hempseed oil, linseed oil, mango kernel oil, meadowfoam oil, neat's foot oil, olive oil, palm oil, palm kernel oil, peanut oil, rapeseed oil, rice bran oil, safflower oil, sasanqua oil, soybean oil, sunflower seed oil, tall oil, tsubaki oil, vegetable oils, marine oils which can be converted into plastic or solid fats such as menhaden, candlefish oil, cod-liver oil, orange roughy oil, pile herd, sardine oil, whale and herring oils, 1,3-dipalmitoyl-2-monooleine (POP), 1(3)-palmitoyl-3(1)-stearoyl-2-monooleine (POSt), 1,3-distearoyl-2-monooleine (StOSt), glycerol, triglyceride, diglyceride, monoglyceride, behenic acid triglyceride, trioleine, tripalmitine, tristearine, palm olein, palm stearin, palm kernel olein, palm kernel stearin and triglycerides of medium chain fatty acids; or, processed partial or fully hydrogenated or fractionated oils thereof.

3. The method of claim 1, wherein said initial substrate comprises esters.

4. The method of claim 3, wherein said esters are selected from the group consisting of wax esters, alkyl esters, methyl esters, ethyl esters, isopropyl esters, octadecyl esters, aryl esters, propylene glycol esters, ethylene glycol esters, 1,2-propanediol esters and 1,3-propanediol esters.

5. The method of claim 3, wherein said esters are formed from the esterification or transesterification of monohydroxyl alcohols or polyhydroxyl alcohols.

6. The method of claim 5, wherein said monohydroxyl alcohols or said polyhydroxyl alcohols are primary, secondary or tertiary alcohols of annular, straight or branched chain compounds.

7. The method of claim 6, wherein said monohydroxyl alcohols are selected from the group consisting of methyl alcohol, isopropyl alcohol, ally alcohol, ethanol, propanol, n-butanol, iso-butanol, sec-butanol, tert-butanol, n-pentanol, iso-pentanol, n-hexanol or octadecyl alcohol.

8. The method of claim 6, wherein said polyhydroxyl alcohols are selected from the group consisting of glycerol, propylene glycol, ethylene glycol, 1,2-propanediol and 1,3-propanediol.

9. The method of claim 1, wherein said initial substrate comprises primary, secondary or tertiary monohydroxyl alcohols of annular, straight or branched chain compounds.

10. The method of claim 9, wherein said monohydroxyl alcohols are selected from the group consisting of methyl alcohol, isopropyl alcohol, ally alcohol, ethanol, propanol, n-butanol, iso-butanol, sec-butanol, tert-butanol, n-pentanol, iso-pentanol, n-hexanol or octadecyl alcohol.

11. The method of claim 1, wherein said initial substrate comprises primary, secondary or tertiary polyhydroxyl alcohols of annular, straight or branched chain compounds.

12. The method of claim 11, wherein said polyhydroxyl alcohols are selected from the group consisting of glycerol, propylene glycol, ethylene glycol, 1,2-propanediol and 1,3-propanediol.

13. The method of claim 1, wherein said initial substrate comprises one or more fatty acids; and wherein said one or more fatty acids are saturated, unsaturated or polyunsaturated.

14. The method of claim 13 wherein said one or more fatty acids comprise carbon chains from 4 to 22 carbons long.

15. The method of claim 14, wherein said fatty acids are selected from the group consisting of palmitic acid, stearic acid, oleic acid, linoleic acid, linolenic acid, arachidonic acid, erucic acid, caproic acid, caprylic acid, capric acid, lauric acid, myristic acid, eicosapentaenoic acid (EPA), docosahexaenoic acid (DHA), 5-eicosenoic acid, butyric acid, γ-linolenic acid and conjugated linoleic acid.

16. The method of claim 1, wherein said one or more types of purification media and said lipase are packed in one or more columns.

17. The method of claim 16 wherein said columns are jacketed columns in which the temperature of said initial substrate, said purified substrate, said one or more types of purification media or said lipase is regulated.

18. The method of claim 1, wherein said purified substrate is prepared by mixing said initial substrate with said one or more types of purification media in a tank for a batch slurry purification reaction or mixing said initial substrate in a series of tanks for a series of batch slurry purification reactions.

19. The method of claim 18, wherein said purified substrate is separated from said one or more types of purification media via filtration, centrifugation or concentration prior to reacting said purified substrate with said lipase.

20. The method of claim 19, further comprising mixing said purified substrate with said lipase in a tank for a batch slurry reaction, or flowing said purified substrate through a column containing said lipase.

21. The method of claim 1, wherein a bed of said one or more types of purification media is placed upon a bed of said lipase within a column.

22. The method of claim 21 wherein said column is a jacketed column in which the temperature of said initial substrate, said purified substrate, said one or more types of purification media or said lipase is regulated.

23. The method of claim 1, wherein said lipase is obtained from a cultured eukaryotic or prokaryotic cell line.

24. The method of claim 1, wherein said lipase is a 1,3-selective lipase.

25. The method of claim 1, wherein said lipase is a non-selective lipase.

26. The method of claim 1, wherein said purification medium is selected from the group consisting of activated carbon, coal activated carbon, wood activated carbon, peat activated carbon, coconut shell activated carbon, natural minerals, processed minerals, montmorillonite, attapulgite, bentonite, palygorskite, Fuller's earth, diatomite, smectite, hormite, quartz sand, limestone, kaolin, ball clay, talc, pyrophyllite, perlite, silica, sodium silicate, silica hydrogel, silica gel, fumed silica, precipitated silica, dialytic silica, fibrous materials, cellulose, cellulose esters, cellulose ethers, microcrystalline cellulose; alumina, zeolite, starches, molecular sieves, previously used immobilized lipase, diatomaceous earth, ion exchange resin, size exclusion chromatography resin, chelating resins, chiral resins, rice hull ash, reverse phase silica, and bleaching clays.

27. The method of claim 1, wherein said purification medium is silica having a surface area from 200 to 750 m²/g, a mesh value from 3 to 425, an average particle size from 4-200 μ, an average pore radius from 20 to 150 Å, and an average pore volume from 0.68 to 1.15 cm³/g.

28. The method of claim 27, wherein said silica is 35-60 mesh with an average pore size of about 60 Å.

29. The method of claim 1, further comprising:
(d) monitoring enzymatic activity by measuring one or more physical properties of said fats or oils after having contacted said lipase;
(e) adjusting the duration of time for which said purified substrate contacts said lipase, or adjusting the temperature of said initial substrate, said purified substrate, said one or more types of purification media or said lipase; and
(f) adjusting the amount and type of said one or more types of purification media in response to changes in said physical properties to optimize said enzymatic activity.

30. The method of claim 29, wherein said one or more physical properties include the Mettler dropping point temperature of said fats or oils.

31. The method of claim 29, wherein said one or more physical properties include the solid fat content temperature profile of said fats or oils.

32. The method of claims 1 wherein said fats or oils produced are 1,3-diglycerides.
